(19) United States
(12) Patent Application Publication
(10) Pub. No.: US 2015/0073255 A1
(43) Pub. Date: Mar. 12, 2015

(54) SCOUT IMAGE SCANNING METHOD AND SYSTEM

(71) Applicant: Shenyang Neusoft Medical Systems Co., Ltd., Shenyang (CN)

(72) Inventors: Jinjun LIU, Shenyang (CN); Gang Yan, Shenyang (CN)

(73) Assignee: Shenyang Neusoft Medical Systems Co., Ltd., Shenyang (CN)

(21) Appl. No.: 14/142,955

(22) Filed: Dec. 30, 2013

(30) Foreign Application Priority Data
Sep. 6, 2013 (CN) 201310404485.6

(51) Int. Cl. A61B 5/00; A61B 6/03; A61B 6/00; A61B 5/055

(52) U.S. Cl. CPC A61B 5/0037; A61B 5/055; A61B 6/032; A61B 6/037; A61B 6/58
USPC 600/407

(57) ABSTRACT

Embodiments provide a scout image scanning method and system. The method includes: selecting a scanning protocol to determine a scanning part of a target object; looking up, in a first lookup table, a corresponding scanning length value based on the scanning part; looking up, in a second lookup table, corresponding scanning parameters based on the scanning part and sign information of the target object; and automatically setting a scout image scanning dosage based on the scanning length and the scanning parameters obtained from above looking up processes, and implementing a scanning process. Efficiency of the scout image scanning processes may be increased, dosages used therein may be reduced and thus reducing risks of hurting the target objects.

### CROSS REFERENCE TO RELATED APPLICATIONS

The present application claims priority to Chinese Patent Application No. 201310404485.6, filed on Sep. 6, 2013 and entitled “SCOUT IMAGE SCANNING METHOD AND SYSTEM”, the entire disclosure of which is incorporated herein by reference.

### FIELD OF THE DISCLOSURE

The present disclosure generally relates to medical image processing, and more particularly, to a scout image scanning method and a scout image scanning system.

### BACKGROUND OF THE DISCLOSURE

Before using a current medical imaging diagnosis system to implement a scanning process, medical workers need to position a patient based on his/her clinical manifestations and personal features. When the patient is at a predetermined position, normally a scout image scanning process is implemented to determine a scanning range and position of a tomography process, such that the medical workers can conduct the tomography process.

While using a conventional CT system, the medical workers may select a corresponding scanning protocol to implement a scout image scanning process after the position of the patient is determined. However, scanning dosages defined in current scanning protocols have fixed values. On one hand, the fixed dosage values, used for patients having a larger size, may result in an unacceptable scout image which is not useful for the medical workers. On the other hand, for patients having a smaller size or juvenile patients, the fixed dosage values may be beyond necessary, which may cause unnecessary radiations to the patients.

### BRIEF SUMMARY OF THE DISCLOSURE

Embodiments of the present disclosure provide a scout image scanning method and a scout image scanning system. Scanning dosage and range suitable for a target object may be automatically determined based on specific sign information of the target object, which may increase scanning efficiency, reduce scanning dosage use and thus reduce hurting the target object from the scanning.

According to one embodiment, an scout image scanning process is provided, the method includes:

selecting a scanning protocol to determine a scanning part of a target object;

looking up, in a first lookup table, a corresponding scanning length value based on the determined scanning part, where the first lookup table is pre-established based on scanning parts, scanning protocols and scanning length values used in clinical practices;

looking up, in a second lookup table, corresponding scanning parameters based on sign information of the target object and the determined scanning part, where the second lookup table is pre-established by analyzing relationships among scanning parts, sign information and standard measurement parameters based on practical clinical scanning results; and

automatically setting a scout image scanning dosage based on the scanning length and the scanning parameters obtained from above looking up processes, and implementing a scanning process.

Optionally, the first lookup table is pre-established by:

recording scanning parts, scanning protocols and scanning length values selected in a plurality of practical clinical scanning processes;

analyzing scanning results of the plurality of practical clinical scanning processes to select a first set of schemes for different scanning parts and scanning protocols, where each of the first set of schemes has a best scanning result and a least scanning length value among schemes for a corresponding scanning part and a corresponding scanning protocol; and

establishing the first lookup table representing relationships among scanning parts, scanning protocols and scanning length values based on the selected first set of schemes.

Optionally, the second lookup table is pre-established by:

recording sign information of target objects, scanning parts and scanning parameters in a plurality of practical clinical scanning processes;

analyzing scanning results of the plurality of practical clinical scanning processes to select a second set of schemes for different scanning parts and sign information, where each of the second set of schemes has a best scanning result and a least scanning parameter value among schemes for a corresponding scanning part and a corresponding piece of sign information; and

establishing the second lookup table representing relationships among scanning parts, sign information and scanning parameter values based on the selected second set of schemes.

Optionally, the sign information includes at least one selected from a group consisting of height, weight and age of the target object.

Optionally, the scanning parameters include tube voltage value, tube current value, or a combination thereof.

According to one embodiment of the present disclosure, a scout image scanning system is provided. The system includes:

a selecting device, adapted for selecting a scanning protocol to determine a scanning part of a target object;

a first establishing device, adapted for pre-establishing a first lookup table based on scanning parts, scanning protocols and scanning length values used in clinical practices;

a second establishing device, adapted for pre-establishing a second lookup table by analyzing relationships among scanning parts, sign information and standard measurement parameters based on practical clinical scanning results;

a first lookup device, adapted for looking up, in the first lookup table, a corresponding scanning length value based on the scanning part;

a second lookup device, adapted for looking up, in the second lookup table, corresponding scanning parameters based on the scanning part and sign information of the target object; and

a setup device, adapted for automatically setting a scout image scanning dosage based on the scanning length and the scanning parameters obtained by the first lookup device and the second lookup device, and initiating a scanning process.

Optionally, the first establishing device includes:

a first recording device, adapted for recording scanning parts, scanning protocols and scanning length values selected in a plurality of practical clinical scanning processes;

a first analyzing device, adapted for analyzing scanning results of the plurality of practical clinical scanning processes to select a first set of schemes for different scanning parts and scanning protocols, where each of the first set of schemes has a best scanning result and a least scanning length value among schemes for a corresponding scanning part and a corresponding scanning protocol; and

a first mapping device, adapted for establishing the first lookup table representing relationships among scanning parts, scanning protocols and scanning length values based on the selected first set of schemes.

Optionally, the second establishing device includes:

a second recording device, adapted for recording sign information of target objects, scanning parts and scanning parameters in a plurality of practical clinical scanning processes;

a second analyzing device, adapted for analyzing scanning results of the plurality of practical clinical scanning processes to select a second set of schemes for different scanning parts and sign information, where each of the second set of schemes has a best scanning result and a least scanning parameter value among schemes for a corresponding scanning part and a corresponding piece of sign information; and

a second mapping device, adapted for establishing the second lookup table representing relationships among scanning parts, sign information and scanning parameter values based on the selected second set of schemes.

Optionally, the sign information includes at least one selected from a group consisting of height, weight and age of the target object.

Optionally, the scanning parameters include tube voltage value, tube current value, or a combination thereof.

Embodiments of the present disclosure provide a scout image scanning method and system, each of which implements: selecting a scanning protocol to determine a scanning part; looking up, in a predefined first lookup table, a corresponding scanning length value based on the scanning part, where the first lookup table is pre-established based on scanning parts, scanning protocols and scanning length values used in clinical practices; looking up, in a second lookup table, corresponding scanning parameters based on the scanning part and sign information of the target object, where the second lookup table is pre-established by analyzing relationships among scanning parts, sign information and standard measurement parameters based on practical clinical scanning results; and automatically setting a scout image scanning dosage based on the scanning length and the scanning parameters obtained from above looking up processes, and implementing a scanning process. Dosages used in scout image scanning processes of embodiments are automatically determined by automatically looking up scanning length values and scanning parameter values based on sign information and specific scanning parts of target objects. Such that, efficiency of the scout image scanning processes may be increased, dosages used therein may be reduced and thus reducing risks of hurting the target objects.

### BRIEF DESCRIPTION OF THE DRAWINGS

In order to clarify the disclosure and advantages thereof, accompanying drawings used in description of embodiments of the present disclosure will be described simply. Obviously, drawings described below are only illustrative and those skilled in the art can obtain other drawings based on these drawings without creative works.

FIG. 1 schematically illustrates a flow chart of a scout image scanning method according to one embodiment of the present disclosure;

FIG. 2 schematically illustrates a flow chart of a process for establishing a first lookup table according to one embodiment of the present disclosure;

FIG. 3 schematically illustrates a flow chart of a process for establishing a second lookup table according to one embodiment of the present disclosure; and

FIG. 4 schematically illustrates a block diagram of a scout image scanning system according to one embodiment of the present disclosure.

### DETAILED DESCRIPTION OF THE DISCLOSURE

In order to clarify the objects, characteristics and advantages of the disclosure, embodiments of the disclosure will be interpreted in detail in combination with accompanied drawings.

It should be noted that methods provided in embodiments of the present disclosure may be used in systems which require a target object to be positioned, such as computed tomography (CT) systems, positron emission tomography (PET) systems, digital radiography (DR) systems, magnetic resonance imaging (MRI) systems, and the like. In such systems, a scout image scanning process is required to be performed to the target object before the systems is started to obtain data by scanning.

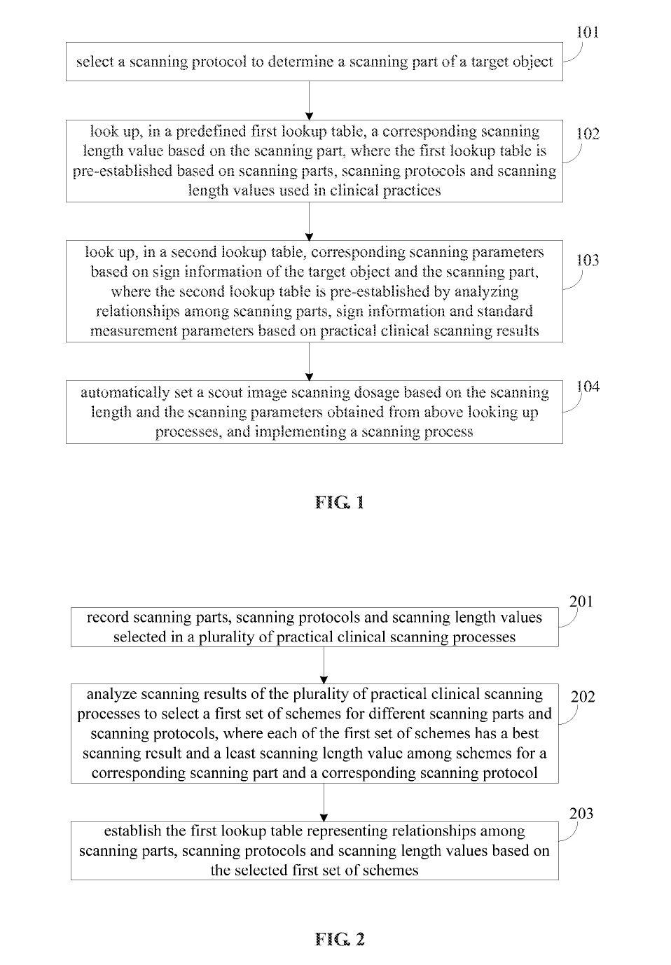

FIG. 1 schematically illustrates a flow chart of a scout image scanning method according to one embodiment of the present disclosure. Referring to FIG. 1, the scout image scanning method may include steps 101, 102, 103 and 104.

In step 101, select a scanning protocol to determine a scanning part of a target object.

In practice, different scanning parts are corresponding to different scanning protocols. Therefore, once a scanning protocol is selected, which part is to be scanned can be determined. For example, a first scanning part 1, which is head, is corresponding to a first scanning protocol 1; a second scanning part 2, which is belly, is corresponding to a second scanning protocol 2; and a third scanning part 3, which is chest, is corresponding to a third scanning protocol 3. Different scanning devices may have different predefined corresponding relationships between scanning protocols and scanning parts. However, the scanning protocols and the scanning parts muse be corresponding to each other one by one.

In step 102, look up, in a predefined first lookup table, a corresponding scanning length value based on the scanning part, where the first lookup table is pre-established based on scanning parts, scanning protocols and scanning length values used in clinical practices.

FIG. 2 schematically illustrates a flow chart of a process for establishing the first lookup table according to one embodiment of the present disclosure. The first lookup table may be established by following steps 201, 202 and 203.

In step 201, record scanning parts, scanning protocols and scanning length values selected in a plurality of practical clinical scanning processes.

In step 202, analyze scanning results of the plurality of practical clinical scanning processes to select a first set of schemes for different scanning parts and scanning protocols, where each of the first set of schemes has a best scanning result and a least scanning length value among schemes for a corresponding scanning part and a corresponding scanning protocol. The first set of schemes may reflect optimum scanning length values for different scanning parts and scanning protocols. For example, for a particular scanning part and particular scanning protocol which are corresponding to each other, scanning results may vary with different scanning length values. The one with the least scanning length value and relatively better result may be selected as one scheme of the first set.

In step 203, establish the first lookup table representing relationships among scanning parts, scanning protocols and scanning length values based on the selected first set of schemes.

An example of the first lookup table is listed as “Table One”, which lists out the selected first set of schemes.

TABLE ONE

| Scanning protocol | Scanning part | Scanning length |
|---|---|---|
| Protocol 1 | Part 1 | 200 mm |
| Protocol 2 | Part 2 | 220 mm |
| Protocol 3 | Part 3 | 230 mm |
| . . . | . . . | . . . |
| Protocol n | Part x | . . . |

Referring back to FIG. 1, in step 103, look up, in a second lookup table, corresponding scanning parameters based on sign information of the target object and the scanning part, where the second lookup table is pre-established by analyzing relationships among scanning parts, sign information and standard measurement parameters based on practical clinical scanning results.

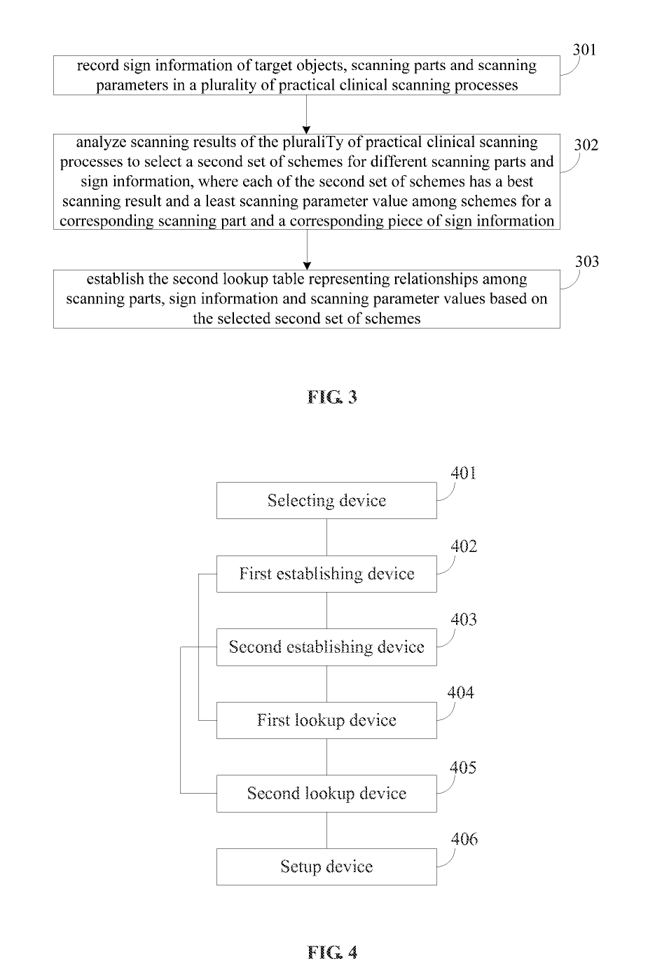

FIG. 3 schematically illustrates a flow chart of a process for establishing the second lookup table according to one embodiment of the present disclosure. The second lookup table may be established by following steps 301, 302 and 303.

In step 301, record sign information of target objects, scanning parts and scanning parameters in a plurality of practical clinical scanning processes.

In step 302, analyze scanning results of the plurality of practical clinical scanning processes to select a second set of schemes for different scanning parts and sign information, where each of the second set of schemes has a best scanning result and a least scanning parameter value among schemes for a corresponding scanning part and a corresponding piece of sign information. The second set of schemes may reflect optimum scanning parameter values, i.e., the standard measurement parameters, for different scanning parts and sign information. For example, for a particular scanning part and particular kind of sign information, scanning results may vary with different scanning parameter values. The one with the least scanning parameter value and relatively better result may be selected as one scheme of the second set.

In step 303, establish the second lookup table representing relationships among scanning parts, sign information and scanning parameter values based on the selected second set of schemes.

In some embodiments, the sign information may include at least one selected from a group consisting of height, weight and age of the target object.

In some embodiments, the scanning parameters may include tube voltage value, tube current value, or a combination thereof.

In some embodiments, if the sign information includes height and weight values, body mass index (BMI) values may be calculated based on the height and weight values. Specifically, one BMI value may be calculated by dividing a weight value (in kilograms) with a square number of a height value (in meters). In such configurations, the second lookup table may list out the selected second set of schemes including scanning parts, BMI values, ages and the selected scanning parameters, i.e., the standard measurement parameters. In some embodiments, the scanning parameters may include tube voltage values and tube current values. An example of the second lookup table established based on the process illustrated in FIG. 3 is listed as “Table Two”, which lists out the selected second set of schemes.

TABLE TWO

| | | | Tube voltage | Tube current |
|---|---|---|---|---|
| Scanning part | BMI range | Age range | (KV) | (MA) |
| Part 1 | 0~15 | 0~20 | 80 | 30 |
| Part 2 | 15~20 | 20~30 | 100 | 30 |
| Part 3 | 20~30 | 30~40 | 120 | 40 |
| . . . | . . . | . . . | . . . | . . . |
| Part x | . . . | . . . | . . . | . . . |

“Table Two” merely illustrates some information of the second lookup table. In practices, the second lookup table may list out various age ranges, different BMI ranges, different scanning parts and their corresponding tube voltage values and tube current values.

In some embodiments, the pre-established second lookup table may include age ranges, BMI ranges, scanning parts and their corresponding tube voltage values, while corresponding tube current values may be set as fixed values in scanning protocols. In some embodiments, the pre-established second lookup table may include age ranges, BMI ranges, scanning parts and their corresponding tube current values, while corresponding tube voltage values may be set as fixed values in scanning protocols.

Referring back to FIG. 1, in step 104, automatically set a scout image scanning dosage based on the scanning length and the scanning parameters obtained from above looking up processes, and implementing a scanning process.

In embodiments described above, the scout image scanning method may include following steps: selecting a scanning protocol to determine a scanning part; looking up, in a predefined first lookup table, a corresponding scanning length value based on the scanning part, where the first lookup table is pre-established based on scanning parts, scanning protocols and scanning length values used in clinical practices; looking up, in a second lookup table, corresponding scanning parameters based on the scanning part and sign information of the target object, where the second lookup table is pre-established by analyzing relationships among scanning parts, sign information and standard measurement parameters based on practical clinical scanning results; and automatically setting a scout image scanning dosage based on the scanning length and the scanning parameters obtained from above looking up processes, and implementing a scanning process. Dosages used in scout image scanning processes of embodiments are automatically determined by automatically looking up scanning length values and scanning parameter values based on sign information and specific scanning parts of target objects. Such that, efficiency of the scout image scanning processes may be increased, dosages used therein may be reduced and thus reducing risks of hurting the target objects.

Embodiments of the present disclosure further provide a scout image scanning system. FIG. 4 schematically illustrates a block diagram of a scout image scanning system according to one embodiment of the present disclosure. Referring to FIG. 4, the system may include: a selecting device 401, a first establishing device 402, a second establishing device 403, a first lookup device 404, a second lookup device 405 and a setup device 406.

Inner structures and interconnections of the system will be illustrated in detail in conjunction with operation principles hereinafter.

The selecting device 401 may be adapted for selecting a scanning protocol to determine a scanning part of a target object.

The first establishing device 402 may be adapted for pre-establishing a first lookup table based on scanning parts, scanning protocols and scanning length values used in clinical practices.

The second establishing device 403 may be adapted for pre-establishing a second lookup table by analyzing relationships among scanning parts, sign information and standard measurement parameters based on practical clinical scanning results.

The first lookup device 404 may be adapted for looking up, in the first lookup table, a corresponding scanning length value based on the scanning part.

The second lookup device 405 may be adapted for looking up, in the second lookup table, corresponding scanning parameters based on the scanning part and sign information of the target object.

The setup device 406 may be adapted for automatically setting a scout image scanning dosage based on the scanning length and the scanning parameters obtained from above looking up processes, and initiating a scanning process.

In some embodiments, the first establishing device may include a first recording device, a first analyzing device and a first mapping device.

The first recording device may be adapted for recording scanning parts, scanning protocols and scanning length values selected in a plurality of practical clinical scanning processes.

The first analyzing device may be adapted for analyzing scanning results of the plurality of practical clinical scanning processes to select a first set of schemes for different scanning parts and scanning protocols, where each of the first set of schemes has a best scanning result and a least scanning length value among schemes for a corresponding scanning part and a corresponding scanning protocol.

The first mapping device may be adapted for establishing the first lookup table representing relationships among scanning parts, scanning protocols and scanning length values based on the selected first set of schemes.

In some embodiments, the second establishing device may include a second recording device, a second analyzing device and a second mapping device.

The second recording device may be adapted for recording sign information of target objects, scanning parts and scanning parameters in a plurality of practical clinical scanning processes.

The second analyzing device may be adapted for analyzing scanning results of the plurality of practical clinical scanning processes to select a second set of schemes for different scanning parts and sign information, where each of the second set of schemes has a best scanning result and a least scanning parameter value among schemes for a corresponding scanning part and a corresponding piece of sign information.

The second mapping device may be adapted for establishing the second lookup table representing relationships among scanning parts, sign information and scanning parameter values based on the selected second set of schemes.

In some embodiments, the sign information may include at least one selected from a group consisting of height, weight and age of the target object.

In some embodiments, the scanning parameters may include tube voltage value, tube current value, or a combination thereof.

In embodiments described above, the scout image scanning system may implement following steps: selecting a scanning protocol to determine a scanning part; looking up, in a predefined first lookup table, a corresponding scanning length value based on the scanning part, where the first lookup table is pre-established based on scanning parts, scanning protocols and scanning length values used in clinical practices; looking up, in a second lookup table, corresponding scanning parameters based on the scanning part and sign information of the target object, where the second lookup table is pre-established by analyzing relationships among scanning parts, sign information and standard measurement parameters based on practical clinical scanning results; and automatically setting a scout image scanning dosage based on the scanning length and the scanning parameters obtained from above looking up processes, and implementing a scanning process. Dosages used in scout image scanning processes of embodiments are automatically determined by automatically looking up scanning length values and scanning parameter values based on sign information and specific scanning parts of target objects. Such that, efficiency of the scout image scanning processes may be increased, dosages used therein may be reduced and thus reducing risks of hurting the target objects.

It should be noted that the scout image scanning system provided in embodiments of the present disclosure may be a separated system or mounted in a current scanning system.

It should be noted that, in descriptions of the present disclosure, terms like “first” and “second” are used merely for distinguishing an entity/operation from another entity/operation, which may not require or suggest that the entities/operations must have substantial relationship or order. Moreover, the term “comprising”, “including” or any other variants thereof are intended to cover a non-exclusive inclusion, such that a number of processes, methods, articles, or devices including certain elements not only include those elements, but also include other elements not explicitly listed, or include inherent elements for such processes, methods, articles or devices. In the case where no more restrictions is illustrated, elements with limitations of “includes a . . . ” do not preclude that there are other same elements included in processes, methods, articles, or devices of the elements.

It should be noted that, those skilled in the art may understand all or some of the processes in the methods described above can be realized by using computer programs to instruct corresponding hardware. The programs may be stored in a readable storage medium in a computer. When the programs are implemented, the processes in the methods in the above embodiments may be performed. The readable storage medium may be diskette, CD (Compact Disc), ROM (Read-Only Memory), RAM (Random Access Memory) or the like.

Systems and methods for scout image scanning are described. Although the present disclosure has been disclosed above with reference to preferred embodiments thereof, it should be understood that the disclosure is presented by way of example only, and not limitation. Those skilled in the art can modify and vary the embodiments without departing from the spirit and scope of the present disclosure.

What is claimed is:

1. A scout image scanning method, comprising:
selecting a scanning protocol to determine a scanning part of a target object;
looking up, in a first lookup table, a corresponding scanning length value based on the determined scanning part, where the first lookup table is pre-established based on scanning parts, scanning protocols and scanning length values used in clinical practices;
looking up, in a second lookup table, corresponding scanning parameters based on sign information of the target object and the determined scanning part, where the second lookup table is pre-established by analyzing relationships among scanning parts, sign information and standard measurement parameters based on practical clinical scanning results; and
automatically setting a scout image scanning dosage based on the scanning length and the scanning parameters obtained from above looking up processes, and implementing a scanning process.

2. The method according to claim 1, wherein the first lookup table is pre-established by:
recording scanning parts, scanning protocols and scanning length values selected in a plurality of practical clinical scanning processes;
analyzing scanning results of the plurality of practical clinical scanning processes to select a first set of schemes for different scanning parts and scanning protocols, where each of the first set of schemes has a best scanning result and a least scanning length value among schemes for a corresponding scanning part and a corresponding scanning protocol; and
establishing the first lookup table representing relationships among scanning parts, scanning protocols and scanning length values based on the selected first set of schemes.

3. The method according to claim 1, wherein the second lookup table is pre-established by:
recording sign information of target objects, scanning parts and scanning parameters in a plurality of practical clinical scanning processes;
analyzing scanning results of the plurality of practical clinical scanning processes to select a second set of schemes for different scanning parts and sign information, where each of the second set of schemes has a best scanning result and a least scanning parameter value among schemes for a corresponding scanning part and a corresponding piece of sign information; and
establishing the second lookup table representing relationships among scanning parts, sign information and scanning parameter values based on the selected second set of schemes.

4. The method according to claim 1, wherein the sign information comprises at least one selected from a group consisting of height, weight and age of the target object.

5. The method according to claim 1, wherein the scanning parameters comprise tube voltage value, tube current value, or a combination thereof.

6. A scout image scanning system, comprising:
a selecting device, adapted for selecting a scanning protocol to determine a scanning part of a target object;
a first establishing device, adapted for pre-establishing a first lookup table based on scanning parts, scanning protocols and scanning length values used in clinical practices;
a second establishing device, adapted for pre-establishing a second lookup table by analyzing relationships among scanning parts, sign information and standard measurement parameters based on practical clinical scanning results;
a first lookup device, adapted for looking up, in the first lookup table, a corresponding scanning length value based on the scanning part;
a second lookup device, adapted for looking up, in the second lookup table, corresponding scanning parameters based on the scanning part and sign information of the target object; and
a setup device, adapted for automatically setting a scout image scanning dosage based on the scanning length and the scanning parameters obtained by the first lookup device and the second lookup device, and initiating a scanning process.

7. The system according to claim 6, wherein the first establishing device comprises:
a first recording device, adapted for recording scanning parts, scanning protocols and scanning length values selected in a plurality of practical clinical scanning processes;
a first analyzing device, adapted for analyzing scanning results of the plurality of practical clinical scanning processes to select a first set of schemes for different scanning parts and scanning protocols, where each of the first set of schemes has a best scanning result and a least scanning length value among schemes for a corresponding scanning part and a corresponding scanning protocol; and
a first mapping device, adapted for establishing the first lookup table representing relationships among scanning parts, scanning protocols and scanning length values based on the selected first set of schemes.

8. The system according to claim 6, wherein the second establishing device comprises:
a second recording device, adapted for recording sign information of target objects, scanning parts and scanning parameters in a plurality of practical clinical scanning processes;
a second analyzing device, adapted for analyzing scanning results of the plurality of practical clinical scanning processes to select a second set of schemes for different scanning parts and sign information, where each of the second set of schemes has a best scanning result and a least scanning parameter value among schemes for a corresponding scanning part and a corresponding piece of sign information; and
a second mapping device, adapted for establishing the second lookup table representing relationships among scanning parts, sign information and scanning parameter values based on the selected second set of schemes.

9. The system according to claim 6, wherein the sign information comprises at least one selected from a group consisting of height, weight and age of the target object.

10. The system according to claim 6, wherein the scanning parameters comprise tube voltage value, tube current value, or a combination thereof.
